(19) United States
(12) Patent Application Publication
(10) Pub. No.: US 2021/0085258 A1
(43) Pub. Date: Mar. 25, 2021

(54) SYSTEM AND METHOD TO PREDICT A STATE OF DROWSINESS IN A SUBJECT

(71) Applicant: PathPartner Technology Private Limited, Bengaluru (IN)

(72) Inventors: Dakala Jayachandra, Bangalore (IN); Kalyan Kumar Hati, Orissa (IN); Mani Kumar Tellamekala, Andhra Pradesh (IN)

(21) Appl. No.: 17/033,358

(22) Filed: Sep. 25, 2020

(30) Foreign Application Priority Data
Sep. 25, 2019 (IN) 201941038804

(51) Int. Cl. A61B 5/00; A61B 5/16; G06K 9/00

(52) U.S. Cl. CPC A61B 5/7275; A61B 5/163; G06K 9/00597; G06K 9/00268; A61B 5/0077

(57) ABSTRACT

The present disclosure provides a system and method for predicting drowsiness of a subject. During facial landmark tracking of a received stream of images, feature extraction is split into two components, viz., landmark independent and landmark dependant features. Precomputing the landmark independent features during a first stage of a first cascade reduces the computational requirements of the approach without affecting accuracy of detection of landmark features. A second multi-stage cascade is used to distinguish and extract eye images from the facial landmarks. At the end of the second multi-stage cascade, the eye landmarks are again distinguished between open eyes and closed eyes using a third multi-stage cascade. Progressively, the subsequent stages of the third multi-stage cascade discards open eyes, and eventually detects closed eyes accurately. The occurrence of closed eye images and eye height variations in the received stream of images can enable determination of drowsiness of the subject.

### CROSS-REFERENCE TO RELATED APPLICATIONS

This application is related to and claims priority to Indian Patent Application No. 201941038804 filed on Sep. 25, 2019, the contents of which are incorporated by reference herein.

### TECHNICAL FIELD

The present disclosure relates generally to the field of eye tracking. In particular, the present disclosure relates to tracking of closed eye, open eye and eye height to predict drowsiness of a subject.

### BACKGROUND

Background description includes information that may be useful in understanding the present invention. It is not an admission that any of the information provided herein is prior art or relevant to the presently claimed invention, or that any publication specifically or implicitly referenced is prior art.

Eye analysis can be an efficient way to monitor drowsiness of a person, especially when the person is about to perform an operation requiring of focus and swift reflex such as driving. Drowsiness and fatigue severely affects driving behaviour and has been shown to be one of the top reasons in many road accidents. Eye analysis typically involves finding the location of the eyes in a given face image, finding the pupil centre and eye ball centre (inferring eye ball centre either from corneal reflections or from the estimating the 3D eye landmarks) for eye gaze, predicting the eye open/close status and computing the eye height for analysing eye blinks and eye closure for drowsiness detection.

There are several indicators that can allow prediction of drowsiness of a person such as slow eyelid closures, increased number and duration of eye blinks, reduced pupil diameter, excessive yawning, head nodding, slowdown of breathing and heart rate, decline of muscle tone and body temperature, electromyogram (EMG) shift to lower frequencies and higher amplitude, increase of electroencephalogram (EEG) alpha waves etc. Analysis of any or all the above-mentioned indicators can provide sufficient clues to predict how drowsy the person is.

A study conducted to determine a correlation between the above-mentioned indicators with the psychomotor vigilance task (PVT), considered to be the true indicator of drowsiness, determined that while indicators such as EEG, head position, and eye blinks are reasonable indicators for some individuals, they are poor indicators for others. However, measures based on percentage of eye closure (PERCLOS) were found to have a consistently high correlation with the PVT measure and hence PERCLOS measures can be considered good drowsiness indicators.

Electro-occulogram (EOG) is considered a high standard for drowsiness. However, EOG capture set-up can be disturbing to drivers. A study has indicated that if blinking rate of a driver is analysed at frame rates of over 100 fps, the results have a strong correlation with results from EOG. In summary, analysing PERCLOS and blink rates above 100 fps can be considered to be effective ways to predict state of drowsiness of a person.

Reliable eye analysis at 100 fps and above involve two major challenges—firstly, the detection of eye and eye landmarks at 100 fps under varying conditions like light variations, presence or absence of spectacles etc., and secondly, the assessment of openness or closure of eye for different eye sizes and shapes. The above challenges get more complex considering that any methodology to analyse the above-mentioned indicators needs to be done at over 100 fps.

Existing solutions for the eye analysis can be broadly grouped into three classes. One class of systems incorporate a sensor and illumination set up that typically aims to produce or capture special reflections from the eyes. This class of systems need special hardware set-up and they typically capture alternate images with different illuminators. Having such varying temporal illumination may limit the effective frame rate of these systems. Generating reflections and estimating the reflections from the images reliably under varying day-night lighting conditions and varying head movements could be a limitation of these systems.

Second class of systems use image processing over simple sensor set-up to detect and track eye region and analyse upper and lower eye lid movement for assessing eye openness and eyes closure. For detecting face and then localizing eye regions, these approaches either rely on model-based approaches or on image processing. Then, for detecting eye lids from eye regions they depend on heuristics. Coming up with heuristics that can generalize across large variations in eye shape among people, with and without spectacles, and varying head movements is challenging task. Detecting the upper and lower eye lids under varying lighting conditions with those heuristics using image processing may be the limitation of these methods. Along with these limitations, these methods would still need to be able to detect faces followed by detection of eye regions and then eye lid analysis within 10 ms to process 100 fps.

Third class of systems use machine learning (ML) and/or deep learning (DL) models with simple sensor set-up to detect the eyes and assess eye openness and eye closure. These methods depend on large scale data sets for training models. The accuracy of these models depend on both the diversity of the data set and the model's complexity to learn problem specific representations. With the availability of large-scale data sets ML/DL approached became the first choice to solve computer vision problems. In general, given sufficient data, these methods can outperform image processing-based approaches. For the given problem the focus has been on designing models for detecting faces, detecting/tracking facial key points like eyes, nose etc., and assessing eye status. Particularly for eye status analysis, some methods develop variety of image descriptors that can capture eye structure effectively, whereas, other methods encode eye structure for say open and closed eye with varying number of key points and then develop models to detect those key points. Again, the key challenge here is achieving robust eye analysis within the minimal computation complexity so that all the analysis can be done at 100 fps.

Patent document CN107704805 discloses a method for detection of drowsiness of a driver in a vehicle during driving using detection of facial features obtained by pre-processing facial images as received from a camera. The method involves obtaining information about a number of key features around the mouth and eyes of the driver to determine the state of drowsiness of the driver. The method further includes the detection of closes eye images and detecting its statistical occurrence to determine drowsiness of the driver.

Patent document CN108647616 discloses a real-time sleepiness detection method based on detection of eye-closing and yawning. Images from a camera is processed to detect sleepiness by monitoring the occurrences of closed eyes and yawns in a statistical manner in a series of images obtained from the camera.

However, the above cited patent documents rely on the detection of closed eyes to determine the state of drowsiness of the driver. The detection of closed eyes however includes the rejection of a larger number of image frames indicating open eyes, which makes the process resource intensive.

Patent document WO2018107979 discloses a method for detection of orientation and pose of a human face using cascade regression techniques. Patent document WO2019033571 discloses a method and device for identifying facial features from real-time facial images. The above disclosed patent documents do not provide a solution to the detection of drowsiness of a driver in a vehicle.

There is, therefore, a requirement in the art for an approach to analyse eye movements to predict a state of drowsiness of a person, the approach being able to analyse eye movements at 100 fps. It is further preferable for the approach to be implemented without the requirement of significant processing resource.

All publications herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

In some embodiments, the numbers expressing quantities or dimensions of items, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all groups used in the appended claims.

### OBJECTS

A general object of the present disclosure is to provide a system and method for detection of a state of drowsiness of a subject.

Another object of the present disclosure is to provide a system and method for accurate tracking of closing and opening of eyes of the subject.

Another object of the present disclosure is to provide a system and method that is simple and does not require large computing resources.

Another object of the present disclosure is to provide a system and method that can be easily implemented.

### SUMMARY

The present disclosure relates generally to the field of eye tracking. In particular, the present disclosure relates to tracking of closed eye, open eye and eye height to predict drowsiness of a subject.

In an aspect, the present disclosure provides a system to predict a state of drowsiness of a subject, said system comprising: a memory operatively coupled to one or more processors, said memory storing instructions executable by the one or more processors to: receive, from one or more cameras operatively coupled to it, a stream of images of a face of the subject; detect a plurality of facial landmarks; extract a plurality of facial features; extract a plurality of eye landmarks; segregate closed eye images; and predict state of drowsiness of the subject.

In another aspect, the plurality of facial landmarks are detected from a first received image of face of the subject, and at a pre-determined template of an area around face of the subject at a predetermined scale.

In another aspect, the detected plurality of facial landmarks are tracked in the subsequent received images of the face of the subject, wherein a first multi-stage cascade is applied to enhance accuracy of tracking of the detected plurality of facial landmarks.

In another aspect, a plurality of location dependant facial features and a plurality of location independent facial features are extracted from a first stage of the cascade, wherein the plurality of location dependant features are computed for subsequent stages of the cascade based on a corresponding tracked plurality of landmarks from a preceding stage of the cascade.

In another aspect, a plurality of eye landmarks from the corresponding plurality of facial landmarks is extracted, wherein second multi-stage cascade is applied to enhance accuracy of extraction of the plurality of eye landmarks to enable separation of eye-images from non-eye images.

In another aspect, closed eye images are segregated from the plurality of eye landmarks, wherein a third multi-stage cascade is applied to enhance accuracy of segregation of closed eye images, the remaining images being discarded to enable detection of occurrence of closed eyes in the received stream of images of the face of the subject.

In another aspect, a blink rate and a blink duration are estimated based on frequency of occurrence of closed eye images in the received stream of images of the face of the subject to enable prediction of state of drowsiness of the subject.

In an embodiment, the one or more optical cameras capture images at a rate not less than 100 frames-per-second.

In another embodiment, the multi-stage cascade is a regression cascade, the regression formulated as a regression matrix, and wherein the regression matrix is converted to a fixed-point precision regression matrix.

In another embodiment, the plurality of facial features extracted is any of histogram of oriented gradients (HOG) and scale invariant features transform (SIFT).

In another embodiment, a boosted cascade is applied to extraction of the plurality of facial features to enhance accuracy of the extracted plurality of facial features.

In another embodiment, eye images and non-eye images are differentiated using normalised pixel differences (NPD).

In an aspect, the present disclosure provides a method to predict a state of drowsiness of a subject, said method comprising the steps of: receiving, from one or more cameras operatively coupled to it, a stream of images of a face of the subject; detecting a plurality of facial landmarks; extracting a plurality of facial features; extracting a plurality of eye landmarks; segregating closed eye images; and predict state of drowsiness of the subject.

In another aspect, the plurality of facial landmarks are detected from a first received image of face of the subject, and at a pre-determined template of an area around face of the subject at a predetermined scale.

In another aspect, the detected plurality of facial landmarks are tracked in the subsequent received images of the face of the subject, wherein a first multi-stage cascade is applied to enhance accuracy of tracking of the detected plurality of facial landmarks.

In another aspect, a plurality of location dependant facial features and a plurality of location independent facial features are extracted from a first stage of the cascade, wherein the plurality of location dependant features are computed for subsequent stages of the cascade based on a corresponding tracked plurality of landmarks from a preceding stage of the cascade.

In another aspect, a plurality of eye landmarks from the corresponding plurality of facial landmarks is extracted, wherein second multi-stage cascade is applied to enhance accuracy of extraction of the plurality of eye landmarks to enable separation of eye-images from non-eye images.

In another aspect, closed eye images are segregated from the plurality of eye landmarks, wherein a third multi-stage cascade is applied to enhance accuracy of segregation of closed eye images, the remaining images being discarded to enable detection of occurrence of closed eyes in the received stream of images of the face of the subject.

In another aspect, a blink rate and a blink duration are estimated based on frequency of occurrence of closed eye images in the received stream of images of the face of the subject to enable prediction of state of drowsiness of the subject.

In an embodiment, the stream of images are received at a rate of not less than 100 frames-per-second.

In another embodiment, the multi-stage cascade is a regression cascade, the regression formulated as a regression matrix, and wherein the regression matrix is converted to a fixed-point precision regression matrix.

In another embodiment, the plurality of facial features extracted is any of histogram of oriented gradients (HOG) and scale invariant features transform (SIFT).

In another embodiment, a boosted cascade is applied to extraction of the plurality of facial features to enhance accuracy of the extracted plurality of facial features.

In another embodiment, eye images and non-eye images are differentiated using normalised pixel differences (NPD).

Various objects, features, aspects and advantages of the inventive subject matter will become more apparent from the following detailed description of preferred embodiments, along with the accompanying drawing figures in which like numerals represent like components.

### BRIEF DESCRIPTION OF DRAWINGS

The accompanying drawings are included to provide a further understanding of the present disclosure and are incorporated in and constitute a part of this specification. The drawings illustrate exemplary embodiments of the present invention and, together with the description, serve to explain the principles of the present disclosure.

FIG. 1 illustrates an exemplary module diagram for a system to predict a state of drowsiness of a subject, in accordance with an embodiment of the present disclosure.

FIG. 2 illustrates a representation of a typical approach for determination if a detected eye is open or closed using a boosted cascaded classifier.

FIG. 3 illustrates an exemplary representation of the proposed approach for determination if a detected eye is open or closed using a boosted cascaded classifier, in accordance with an embodiment of the present disclosure.

FIG. 4 illustrates an exemplary flow diagram for a method to predict a state of drowsiness of a subject, in accordance with an embodiment of the present disclosure.

FIG. 5 illustrates an exemplary flow diagram for the proposed method to predict a state of drowsiness of a subject, in accordance with an embodiment of the present disclosure.

FIG. 6 illustrates a computer system in which or with which embodiments of the present invention can be utilized in accordance with embodiments of the present disclosure.

### DETAILED DESCRIPTION

The following is a detailed description of embodiments of the disclosure depicted in the accompanying drawings. The embodiments are in such detail as to clearly communicate the disclosure. However, the amount of detail offered is not intended to limit the anticipated variations of embodiments; on the contrary, the intention is to cover all modifications, equivalents, and alternatives falling within the spirit and scope of the present disclosure as defined by the appended claims.

If the specification states a component or feature “may”, “can”, “could”, or “might” be included or have a characteristic, that particular component or feature is not required to be included or have the characteristic.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise.

Exemplary embodiments will now be described more fully hereinafter with reference to the accompanying drawings, in which exemplary embodiments are shown. These exemplary embodiments are provided only for illustrative purposes and so that this disclosure will be thorough and complete and will fully convey the scope of the invention to those of ordinary skill in the art. The invention disclosed may, however, be embodied in many different forms and should not be construed as limited to the embodiments set forth herein. Various modifications will be readily apparent to persons skilled in the art. The general principles defined herein may be applied to other embodiments and applications without departing from the spirit and scope of the invention. Moreover, all statements herein reciting embodiments of the invention, as well as specific examples thereof, are intended to encompass both structural and functional equivalents thereof. Additionally, it is intended that such equivalents include both currently known equivalents as well as equivalents developed in the future (i.e., any elements developed that perform the same function, regardless of structure). Also, the terminology and phraseology used is for the purpose of describing exemplary embodiments and should not be considered limiting. Thus, the present invention is to be accorded the widest scope encompassing numerous alternatives, modifications and equivalents consistent with the principles and features disclosed. For purpose of clarity, details relating to technical material that is known in the technical fields related to the invention have not been described in detail so as not to unnecessarily obscure the present invention.

The use of any and all examples, or exemplary language (e.g., “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Embodiments described herein relate generally to the field of eye tracking and, in particular, to tracking of closed eye and open eye to predict drowsiness of a subject.

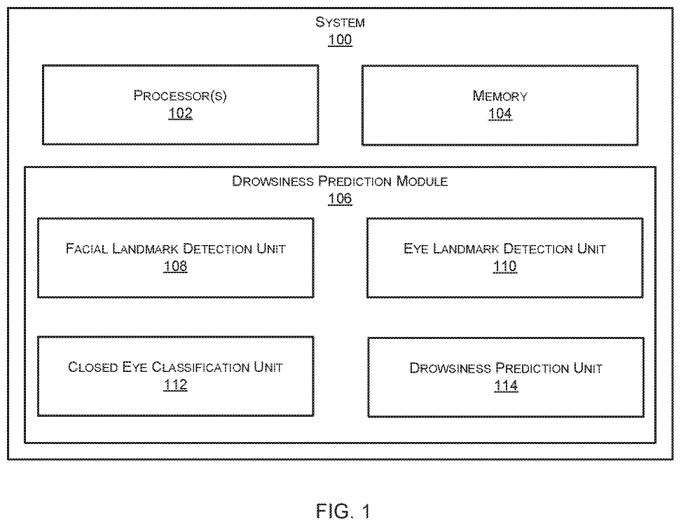

FIG. 1 illustrates an exemplary module diagram for a system to predict a state of drowsiness of a subject, in accordance with an embodiment of the present disclosure. In an embodiment, the system 100 comprises: one or more processors 102 operatively coupled with a memory 104, the memory 104 storing instruction executable by the one or more processors 102 to detect a state of drowsiness of a subject.

In another embodiment, the system 100 comprises a drowsiness prediction module 106 further comprising: a facial landmark detection unit 108; eye landmark detection unit 110; a closed eye classification unit 112; and a drowsiness prediction unit 114.

In another embodiment, the facial landmark detection unit 108 is configured to receive a stream of images of the subject from one or more cameras operatively coupled to it. The images can pertain to an area where the face of the subject is prominently featured. In an exemplary embodiment, the one or more cameras can be any or a combination of infra-red cameras and RGB cameras.

In another embodiment, to increase accuracy of facial landmarks detection, a regression method is applied on the detected facial landmarks such as any known in the art. In an exemplary embodiment, cascading is applied on the extracted features. For application of the regression method, facial landmarks are detected in a first frame of the stream of images, and in subsequent stages of the cascade, the facial landmarks are tracked, and their accuracy improves with each state of the cascade. The accuracy of the detection of facial landmarks depends on the type of landmark, the number of landmarks and the number of cascades. The more numbers for the above stated parameters, higher is the accuracy of detection. However, this comes at a price of increased complexity of the method and increased requirement of processing resources. In an exemplary embodiment, the number of landmarks used can be around twenty-nine, but not exceeding sixty-eight.

In another embodiment, the images are analysed to extract facial features such as by extracting shape indexed features. The features can be local binary features (LBF), a histogram of oriented gradients (HOG), scale invariant features transform (SIFT) or similar such features that can be extracted for analysis, as known in the art. In another embodiment, deep learning methodologies and techniques can also be used for extraction of features.

In another embodiment, each cascade improves accuracy of detection and tracking of the facial landmarks. The refined landmarks from a stage of the cascade are used for further refinement of landmarks in subsequent stages of cascade. While this method improves accuracy, it also requires large amounts of processing resource in order to cascade multiple facial landmarks.

For the case of detection of drowsiness, the landmark area of interest on the face is around the eyes of the subject, and hence, the system 100 utilises a predefined template of an area around the face of the subject, at a predefined scale, and applies the cascades in the predefined template. This can result in reduced requirement of processing resources due to reduced number of data points for processing.

In another embodiment, the regression on feature for landmark location can be formulated as a matrix multiplication. Typically, low-end embedded processors have fixed precision single instruction-multiple data (SIMD) computation, and in order to make computations on them more efficient, after training each cascade, the regression matrix can be converted from a floating-point precision to a fixed-point precision. The fixed-point regression matrix can be used to train a subsequent cascade, where any loss incurred due to fixed-point conversion can be compensated during subsequent cascades.

In another embodiment, the HOG features extracted are classified into two components: a component that can be computed independent of landmark locations; and a component that required landmark locations for its computation.

In another embodiment, the landmark independent features can be implemented on any architecture with vector processing capabilities like single instruction-multiple data (SIMD) operations. The landmark independent features are computed once for a given template in a frame, after which the landmark dependant features are, to a greater extent, computed for every cascade. This selective computing of landmark dependant and landmark independent features further reduces complexity of the system.

In another embodiment, the eye landmark detection unit 110 is configured to focus more exclusively on landmark features pertaining to the eyes of the subject. Facial landmark detection including eye landmarks notwithstanding, the facial landmarks detection unit 108 is configured to focus more on overall face shape retention as opposed to the eye landmarks detection unit 110 which is configured to more precisely detect eye landmarks.

In some instances, partial or full occlusion of an eye can occur due to variations in orientation of the face, in which cases eye landmarks can be outside the face boundary. It is imperative that such instances are not included in further eye analysis as it can result in inaccurate predictions of drowsiness of eyes.

In another embodiment, the eye landmark detection unit 110 is configured to accurately differentiate eye from other background image or faulty image and then to refine the presence of features at eye landmark locations.

In another embodiment, a joint detection and landmark alignment approach is adopted for eye detection against background regions and for eye landmark refinement. In this approach, an image for eye detection is limited to a face region of the subject. This results in checking for eye and non-eye regions within a smaller area of interest, thereby reducing complexity of the approach.

In another embodiment, an approach used to differentiate eye region from non-eye regions is by using normalised pixel differences (NPD). NDP is demonstrated to be efficient under varying illuminations.

In another embodiment, a second cascade can be applied for the detection of eye landmarks. The refined eye landmarks of one stage of the cascade can be used for further refining the tracking of eye landmarks in the subsequent stages.

In another embodiment, the closed eye classification unit 112 is configured to determine if a detected eye is open or closed. Given the variations in eye size and height across the population, as well as eye appearances with changing head poses and the presence or absence of spectacles, the problem of determining if an eye is open or closed can be challenging.

Typically, deep learning-based approaches can be used which can learn complex feature representations required for the problem of determination of whether an eye is open or closed. However, embedded processors, with their limited resources, do not possess the processing capability to handle such a load of data.

Another approach generally used to determine if an eye is open or closed is to enhance predefined feature extractors such as HOG and develop a boosted cascaded classifier.

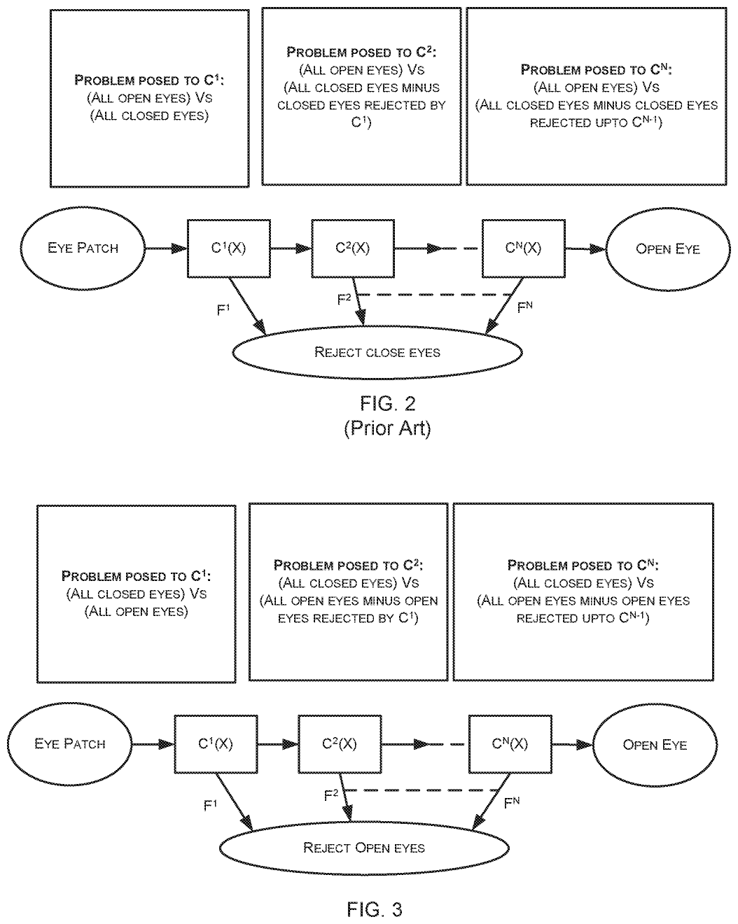

FIG. 2 illustrates a representation of a typical approach for determination if a detected eye is open or closed using a boosted cascaded classifier. A boosted cascade applied once on a refined eye region can be computationally efficient. However, in current implementations of the approach, boosted cascade classifiers are required to retain open eyes by end of the cascade and reject closed eyes in between cascades. An issue arising with such an implementation is that each cascade in the model is required to learn to differentiate between all open eyes versus the remaining closed eyes. The open eyes here can include open eyes with different openness factor which can depend on the subject. Learning cascades which is capable of differentiating between closed eyes against small eyes and partially open eyes can be difficult, and due to this, devising a generalised system to predict drowsiness of a subject that can distinguish between open, partially open and closed eyes for different eye sizes and different head poses is a challenge.

In an aspect, the present disclosure provides an approach to predict drowsiness in a subject by rejecting open eye in each cascade and retain the closed eyes.

FIG. 3 illustrates an exemplary representation of the proposed approach for determination if a detected eye is open or closed using a boosted cascaded classifier, in accordance with an embodiment of the present disclosure. In an embodiment, the model is required to learn to differentiate between fully open eyes and closed eyes. Once fully open eyes are detected and subsequently rejected over a stage of the cascade, the remaining stages of the cascades are left with only requiring learning to differentiate between closed eyes and a set of images pertaining to non-closed eyes that are left over from the preceding cascade. In this way, the problem posed to each subsequent cascade is reduced and the model learns to reject the open and the partially open eyes and retain the closed eyes.

In an exemplary embodiment, the proposed approach uses HOG computed at refined eye landmarks as a feature vector and gradient boosted classifier learnt over such features as a weak classifier.

Referring again to FIG. 1, the closed eye classification unit 112, based on detection of closed eyes, is configured to determine blink rate as the incidence of a closed eye in between a series of open and partially open eyes. The closed eye classification unit 112 is further configured to determine the duration of each blink.

In another embodiment, the drowsiness prediction unit 114 is determined predict drowsiness of the subject based on the blink rate and estimated duration of blink of the subject.

In an aspect, the proposed system for predicting drowsiness of the subject is made less complex by adopting an approach to retain closed eye and reject open and partially open eyes. For facial landmark tracking, the HOG feature extraction is split into two components, viz., landmark independent and landmark dependant features. Precomputing the landmark independent features during a first stage of the cascade reduces the computational requirements of the approach without affecting accuracy of detection of landmark features. Further, regression matrix for facial landmark tracking is converted from a floating-point precision to a fixed-point precision using a fixed-point regression matrix to teach subsequent stages of the cascade, thereby allowing the implementation of regression matrix efficiently without significant loss in accuracy. For detecting eye landmarks features, a simple feature such as NDP is used instead of complex approaches such as HOG to further reduce the computational requirement of the system.

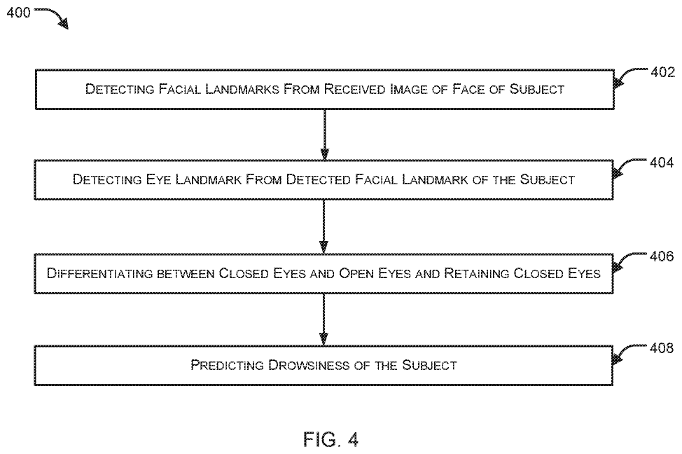

FIG. 4 illustrates an exemplary flow diagram for a method to predict a state of drowsiness of a subject, in accordance with an embodiment of the present disclosure. In an embodiment, the method 400 comprises the steps of: detecting facial landmarks in a subject (402); detecting eye landmarks from the detected facial landmarks (404); segregating closed eyes from open eyes (406); and predicting drowsiness of the subject (408).

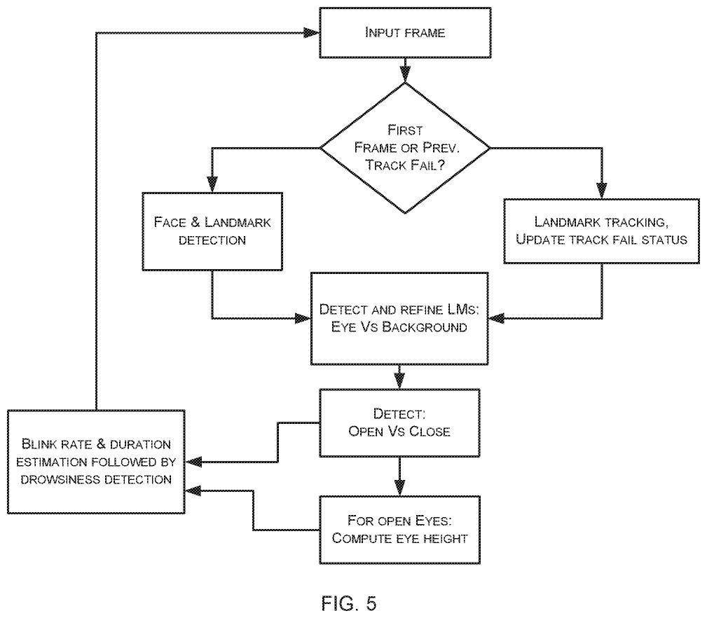

FIG. 5 illustrates an exemplary flow diagram for the proposed method to predict a state of drowsiness of a subject, in accordance with an embodiment of the present disclosure.

In an embodiment, to increase accuracy of facial landmarks detection, a regression method is applied on the detected facial landmarks such as any known in the art. In an exemplary embodiment, cascading is applied on the extracted features. For application of the regression method, facial landmarks are detected in a first frame of the stream of images. In subsequent stages of the cascade, the facial landmarks are tracked, and their accuracy is improved. For each stage of the cascade, the facial landmark tracked in a previous stage of the cascade is used as a template for further refinement, thereby improving accuracy of detection of the facial landmarks with each stage of the cascade. The accuracy of the detection of facial landmarks depends on the type of landmark, the number of landmarks and the number of cascades. The more numbers for the above stated parameters, higher is the accuracy of detection. However, this comes at a price of increased complexity of the method and increased requirement of processing resources. In an exemplary embodiment, the number of landmarks used can be around twenty-nine, but not exceeding sixty-eight.

In another embodiment, a stream of images received of the subject from one or more optical cameras are analysed to extract facial landmarks such as by extracting shape indexed features. The features can be local binary features (LBF), a histogram of oriented gradients (HOG), scale invariant features transform (SIFT) or similar such features that can be extracted for analysis, as known in the art. In another embodiment, deep learning methodologies and techniques can also be used for extraction of features.

In another embodiment, each cascade improves accuracy of detection and tracking of the facial landmarks. The refined landmarks from a stage of the cascade are used for further refinement of landmarks in subsequent stages of cascade. While this method improves accuracy, it also requires large amounts of processing resource in order to cascade multiple facial landmarks.

For the case of detection of drowsiness, the landmark area of interest on the face is around the eyes of the subject, and hence, a predefined template of an area around the eyes of the subject, at a predefined scale is utilised, and the cascades are applied in the predefined template to detect eyes against non-eye areas. Such confinement of background area only around the face can result in reduced requirement of processing resources due to reduced variations of background areas.

In another embodiment, the regression on feature for landmark location can be formulated as a matrix multiplication. In order to make computations more efficient, after training each cascade, the regression matrix can be converted from a floating-point precision to a fixed-point precision. The fixed-point regression matrix can be used to train a subsequent cascade, where any loss incurred due to fixed-point conversion can be compensated during subsequent cascades.

In another embodiment, the HOG features extracted are classified into two components: a component that can be computed independent of landmark locations; and a component that required landmark locations for its computation.

In another embodiment, the landmark independent features are computed once for a given template in a frame, after which the landmark dependant features are, to a greater extent, computed for every cascade. This selective computing of landmark dependant and landmark independent features further reduces complexity of the system.

In another embodiment, the eye landmarks are detected from the facial landmarks of the subject pertaining to the eyes of the subject. In another embodiment, the eye landmark are detected accurately and differentiated from other background image or faulty image and then to refine the presence of features at eye landmark locations during different stages of the cascade.

In another embodiment, a joint detection and landmark alignment approach is adopted for eye detection against background regions and for eye landmark refinement. In this approach, an image for eye detection is limited to a face region of the subject. This results in checking for eye and non-eye regions within a smaller area of interest, thereby reducing complexity of the approach.

In another embodiment, an approach used to differentiate eye region from non-eye regions is by using normalised pixel differences (NPD). NDP is demonstrated to be efficient under varying illuminations.

In another embodiment, it is determined if a detected eye is open or closed. Given the variations in eye size and height across the population, as well as eye appearances with changing head poses and the presence or absence of spectacles, the problem of determining if an eye is open or closed can be challenging.

An approach generally used to determine if an eye is open or closed is to enhance predefined feature extractors such as HOG and develop a boosted cascaded classifier.

In an embodiment, the model is required to learn to differentiate between fully open eyes and closed eyes. Once fully open eyes are detected and subsequently rejected over a stage of the cascade, the remaining stages of the cascades are left with only requiring learning to differentiate between closed eyes and a set of images pertaining to non-closed eyes that are left over from the preceding cascade. In this way, the problem posed to each subsequent cascade is reduced and the model learns to reject the open and the partially open eyes and retain the closed eyes.

In an exemplary embodiment, the proposed approach uses HOG computed at refined eye landmarks as a feature vector and gradient boosted classifier learnt over such features as a weak classifier.

In another embodiment, based on detection of closed eyes, is configured to determine blink rate as the incidence of a closed eye in between a series of open and partially open eyes, along with an estimated duration of each blink. The drowsiness of the subject is predicted based on the blink rate and estimated duration of blink of the subject.

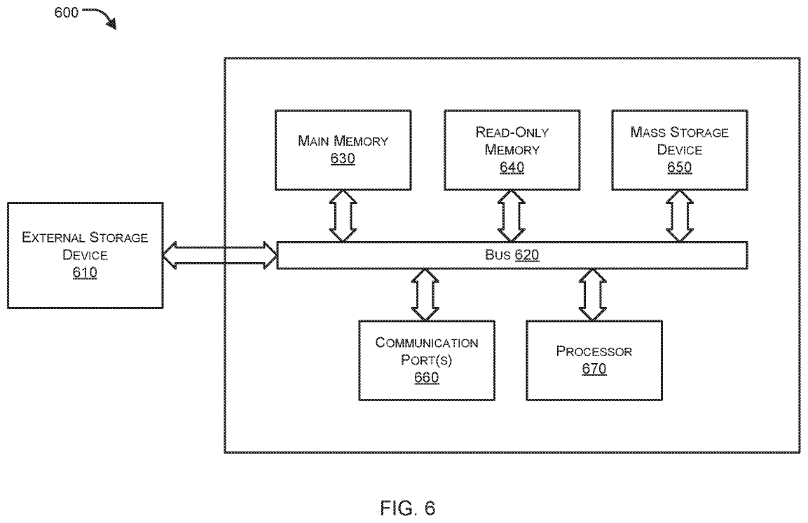

FIG. 6 illustrates a computer system in which or with which embodiments of the present invention can be utilized in accordance with embodiments of the present disclosure.

As shown in FIG. 6, computer system includes an external storage device 610, a bus 620, a main memory 630, a read only memory 640, a mass storage device 650, communication port 660, and a processor 670. A person skilled in the art will appreciate that computer system may include more than one processor and communication ports. Examples of processor 670 include, but are not limited to, an Intel® Itanium® or Itanium 2 processor(s), or AMD® Opteron® or Athlon MP® processor(s), Motorola® lines of processors, FortiSOC™ system on a chip processors or other future processors. Processor 670 may include various modules associated with embodiments of the present invention. Communication port 660 can be any of an RS-232 port for use with a modem-based dialup connection, a 10/100 Ethernet port, a Gigabit or 10 Gigabit port using copper or fibre, a serial port, a parallel port, or other existing or future ports. Communication port 660 may be chosen depending on a network, such a Local Area Network (LAN), Wide Area Network (WAN), or any network to which computer system connects.

Memory 630 can be Random Access Memory (RAM), or any other dynamic storage device commonly known in the art. Read only memory 640 can be any static storage device(s) e.g., but not limited to, a Programmable Read Only Memory (PROM) chips for storing static information e.g., start-up or BIOS instructions for processor 670. Mass storage 650 may be any current or future mass storage solution, which can be used to store information and/or instructions. Exemplary mass storage solutions include, but are not limited to, Parallel Advanced Technology Attachment (PATA) or Serial Advanced Technology Attachment (SATA) hard disk drives or solid-state drives (internal or external, e.g., having Universal Serial Bus (USB) and/or Firewire interfaces), e.g. those available from Seagate (e.g., the Seagate Barracuda 7200 family) or Hitachi (e.g., the Hitachi Deskstar 7K1000), one or more optical discs, Redundant Array of Independent Disks (RAID) storage, e.g. an array of disks (e.g., SATA arrays), available from various vendors including Dot Hill Systems Corp., LaCie, Nexsan Technologies, Inc. and Enhance Technology, Inc.

Bus 620 communicatively couples processor(s) 670 with the other memory, storage and communication blocks. Bus 620 can be, e.g. a Peripheral Component Interconnect (PCI)/PCI Extended (PCI-X) bus, Small Computer System Interface (SCSI), USB or the like, for connecting expansion cards, drives and other subsystems as well as other buses, such a front side bus (FSB), which connects processor 670 to software system.

Optionally, operator and administrative interfaces, e.g. a display, keyboard, and a cursor control device, may also be coupled to bus 620 to support direct operator interaction with computer system. Other operator and administrative interfaces can be provided through network connections connected through communication port 660. External storage device 610 can be any kind of external hard-drives, floppy drives, IOMEGA® Zip Drives, Compact Disc-Read Only Memory (CD-ROM), Compact Disc-Re-Writable (CD-RW), Digital Video Disk-Read Only Memory (DVD-ROM). Components described above are meant only to exemplify various possibilities. In no way should the aforementioned exemplary computer system limit the scope of the present disclosure.

It should be apparent to those skilled in the art that many more modifications besides those already described are possible without departing from the inventive concepts herein. The inventive patent matter, therefore, is not to be restricted except in the spirit of the appended claims. Moreover, in interpreting both the specification and the claims, all terms should be interpreted in the broadest possible manner consistent with the context. In particular, the terms “includes” and “including” should be interpreted as referring to elements, components, or steps in a non-exclusive manner, indicating that the referenced elements, components, or steps may be present, or utilized, or combined with other elements, components, or steps that are not expressly referenced. Where the specification claims refer to at least one of something selected from the group consisting of A, B, C . . . and N, the text should be interpreted as requiring only one element from the group, not A plus N, or B plus N, etc. The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practised with modification within the spirit and scope of the appended claims.

While the foregoing describes various embodiments of the invention, other and further embodiments of the invention may be devised without departing from the basic scope thereof. The scope of the invention is determined by the claims that follow. The invention is not limited to the described embodiments, versions or examples, which are included to enable a person having ordinary skill in the art to make and use the invention when combined with information and knowledge available to the person having ordinary skill in the art.

### ADVANTAGES

The present disclosure provides a system and method for detection of a state of drowsiness of a subject.

The present disclosure provides a system and method for accurate tracking of closing and opening of eyes of the subject.

The present disclosure provides a system and method that is simple and does not require large computing resources.

The present disclosure provides a system and method that can be easily implemented.

We claim:

1. A system to predict a state of drowsiness of a subject, said system comprising:
a memory operatively coupled to one or more processors, said memory storing instructions executable by the one or more processors to:
receive, from one or more cameras operatively coupled to it, a stream of images of a face of the subject;
detect, from a first received image of face of the subject, a plurality of facial landmarks and at a pre-determined template of an area around face of the subject at a predetermined scale;
track, in the subsequent received images of the face of the subject, the detected plurality of facial landmarks, wherein a first multi-stage cascade is applied to enhance accuracy of tracking of the detected plurality of facial landmarks;
extract, from a first stage of the cascade, a plurality of location dependant facial features and a plurality of location independent facial features, wherein the plurality of location dependant features is computed for subsequent stages of the cascade based on a corresponding tracked plurality of landmarks from a preceding stage of the cascade;
extract a plurality of eye landmarks from the corresponding plurality of facial landmarks, wherein a second multi-stage cascade is applied to enhance accuracy of extraction of the plurality of eye landmarks to enable separation of eye-images from non-eye images; and
segregate, from the plurality of eye landmarks, closed eye images, wherein a third multi-stage cascade is applied to enhance accuracy of segregation of closed eye images, the remaining images being discarded to enable detection of occurrence of closed eyes in the received stream of images of the face of the subject, and
wherein a blink rate and a blink duration are estimated based on frequency of occurrence of closed eye images in the received stream of images of the face of the subject to enable prediction of state of drowsiness of the subject.

2. The system as claimed in claim 1, wherein the one or more optical cameras capture images at a rate not less than 100 frames-per-second.

3. The system as claimed in claim 1, wherein the multi-stage cascade is a regression cascade, the regression formulated as a regression matrix, and wherein the regression matrix is converted to a fixed-point precision regression matrix.

4. The system as claimed in claim 1, wherein the plurality of facial features extracted is any of histogram of oriented gradients (HOG) and scale invariant features transform (SIFT).

5. The system as claimed in claim 1, wherein a boosted cascade is applied to extraction of the plurality of facial features to enhance accuracy of the extracted plurality of facial features.

6. The system as claimed in claim 1, wherein eye images and non-eye images are differentiated using normalised pixel differences (NPD).

7. A method to predict a state of drowsiness of a subject, said method comprising the steps of:
receiving, from one or more cameras operatively coupled to it, a stream of images of a face of the subject;
detecting, from a first received image of face of the subject, a plurality of facial landmarks and at a pre-determined template of an area around face of the subject at a predetermined scale;
tracking, in the subsequent received images of the face of the subject, the detected plurality of facial landmarks, wherein a first multi-stage cascade is applied to enhance accuracy of tracking of the detected plurality of facial landmarks;
extracting, from a first stage of the cascade, a plurality of location dependant facial features and a plurality of location independent facial features, wherein the plurality of location dependant features is computed for subsequent stages of the cascade based on a corresponding tracked plurality of landmarks from a preceding stage of the cascade;
extracting a plurality of eye landmarks from the corresponding plurality of facial landmarks, wherein a second multi-stage cascade is applied to enhance accuracy of extraction of the plurality of eye landmarks to enable separation of eye-images from non-eye images; and
segregating, from the plurality of eye landmarks, closed eye images, wherein a third multi-stage cascade is applied to enhance accuracy of segregation of closed eye images, the remaining images being discarded to enable detection of occurrence of closed eyes in the received stream of images of the face of the subject, and
wherein, a blink rate and a blink duration are estimated based on frequency of occurrence of closed eye images in the received stream of images of the face of the subject to enable prediction of state of drowsiness of the subject.

8. The method as claimed in claim 7, wherein the stream of images are received at a rate of not less than 100 frames-per-second.

9. The method as claimed in claim 7, wherein the multi-stage cascade applied is a regression cascade, the regression formulated as a regression matrix, and wherein the regression matrix is converted to a fixed-point precision regression matrix.

10. The method as claimed in claim 7, wherein the plurality of facial features extracted is any of histogram of oriented gradients (HOG) and scale invariant features transform (SIFT).

11. The method as claimed in claim 7, wherein a boosted cascade is applied to extraction of the plurality of facial features to enhance accuracy of the extracted plurality of facial features.

12. The method as claimed in claim 7, wherein eye images and non-eye images are differentiated using normalised pixel differences (NPD).
